(19) United States
(12) Patent Application Publication
(10) Pub. No.: US 2023/0321305 A1
(43) Pub. Date: Oct. 12, 2023

(54) ESSENTIAL OIL DIFFUSION USING DEVICE

(71) Applicant: Chongqing Chuanghong E-Commerce Co., Ltd., Chongqing (CN)

(72) Inventor: Jinhua FANG, Meishan (CN)

(21) Appl. No.: 17/743,283

(22) Filed: May 12, 2022

(30) Foreign Application Priority Data
Apr. 11, 2022 (CN) 2022208292506

(51) Int. Cl. A61L 9/14

(52) U.S. Cl. CPC A61L 9/14; A61L 2209/134; A61L 2209/15

(57) ABSTRACT

An essential oil diffusion using device is provided and includes: a housing, a water tank is arranged in the housing, a support frame is arranged in the water tank, an essential oil pipe is arranged at a side of the support frame, a water stopping sheet is arranged at another side of the support frame, an atomizing sheet is arranged at the another side of the support frame, a middle holder is arranged below the support frame, a fan is arranged on the middle holder, a power supply box is arranged above the fan, an atomization control circuit board is arranged between the power supply box and a top of the middle holder, an atomization circuit is arranged on the atomization control circuit board. The device can achieve diffusion delaying of essential oil, make smell diffusion of an aromatherapy machine more lasting, easy to use, safe and reliable.

### TECHNICAL FIELD

The disclosure relates to the field of essential oil diffusion technologies, and more particularly to an essential oil diffusion using device.

### BACKGROUND

At present, a conventional method of adding essential oil on an aromatherapy machine is to directly pour the essential oil into a water tank, and the water and the essential oil are dispersed into fog through an atomizer. Because the essential oil floats on a surface of the water, it is easy to cause smell of the aromatherapy machine to be thick at the beginning, and then the smell, i.e., the use effect, will become weaker and weaker.

Therefore, consumers hope to increase a cavity of the essential oil to make the essential oil slowly overflow into the water tank, so as to delay the diffusion of the essential oil and make the smell diffusion of the aromatherapy machine more lasting.

Chinese Patent Application No. CN 201720598217.6 (corresponding to Chinese patent publication No. CN 206827350 U), filed on May 25, 2017 and published on Jan. 2, 2018, with a title of essential oil diffuser, which discloses that an essential oil diffuser includes an essential oil bottle, a sealing member and an airflow generating device, the essential oil bottle have a cavity and an opening connected to the cavity, the sealing member is arranged on the opening of the essential oil bottle, the sealing member is provided with an inlet port connected to the cavity and an outlet port connected to the cavity, the airflow generating device is connected to the sealing member and the inlet port. This technical scheme can effectively avoid thicker atomizing granule appearing, has saved the essential oil quantity.

The above Patent Application provides the essential oil diffuser, which cannot meet needs of the consumers because the smell concentration of the emitted essential oil is unbalanced, the effect of delaying the diffusion of the essential oil is not very good.

### SUMMARY

The main purpose of the disclosure is to provide an essential oil diffusion using device with reasonable structure, which can delay the diffusion of essential oil, make the smell diffusion of ab aromatherapy machine more lasting, easy to use, safe and reliable.

In order to realize the purpose of the disclosure, the disclosure provides an essential oil diffusion using device including a housing, a water tank is arranged in the housing, a support frame is arranged in the water tank, an essential oil pipe is arranged at a side of the support frame, a water stopping sheet is arranged at another side of the support frame, an atomizing sheet is arranged at a bottom of the another side of the support frame, a middle holder is arranged below the support frame, a fan is arranged at a bottom of the middle holder, a power supply box is arranged at a top of the fan, an atomization control circuit board is arranged between the power supply box and a top of the middle holder, and an atomization circuit is arranged on the atomization control circuit board.

In an embodiment, a top of the water stopping sheet is provided with a water stopping cover.

In an embodiment, the side of the support frame is provided with an essential oil slot, and an end of the essential oil pipe is arranged in the essential oil slot.

In an embodiment, a bottom of the support frame is provided with a support rod, and the support frame is connected to the middle holder through the support rod.

In an embodiment, a base is arranged at the bottom of the middle holder and is detachably connected to the housing.

In an embodiment, the atomization circuit includes a power supply circuit, a microcontroller unit (MCU) control circuit, an atomization driving circuit, an atomization control circuit and a fan control circuit; the power supply circuit is configured to provide a power to the MCU control circuit, the MCU control circuit is configured to control the atomization control circuit through the atomization driving circuit, the atomization control circuit is configured to control the atomization sheet, and the MCU control circuit is further configured to control the fan control circuit.

In an embodiment, the MCU control circuit includes a water level detection circuit.

The technical solutions of the present disclosure are to make the essential oil slowly overflow into the water tank by designing the essential oil cavity (also referred to as essential oil pipe) in the housing, thereby to delay the diffusion of the essential oil and make the smell diffusion of the aromatherapy machine more lasting. The disclosure has reasonable structure, low cost, safe and reliable use. The disclosure can be used not only in the essential oil diffusion device, but also in ultrasonic atomization products such as a humidifier, which has a wide range of application and is suitable for general promotion.

### BRIEF DESCRIPTION OF DRAWINGS

In order to more clearly explain embodiments of the disclosure or the technical solutions in the related art, the following will briefly introduce the attached drawings that need to be used in the embodiments or the description of the related art. It is obvious that the attached drawings in the following description are only some embodiments of the disclosure. For those skilled in the art, without paying creative labor, other drawings can also be obtained according to the structure shown in these drawings.

FIG. 1 is a schematic structural view of an essential oil diffusion using device according to an embodiment of the disclosure.

FIG. 2 is a schematic circuit block view of an atomization control circuit of an essential oil diffusion using device according to another embodiment of the disclosure.

### DESCRIPTION OF REFERENCE NUMERALS

1-housing; 2-water stopping cover; 3-essential oil pipe; 4-water tank; 41-support frame; 411-essential oil slot; 412-support rod; 5-middle holder; 6-base; 7-water stopping sheet; 8-atomizing sheet; 9-atomization control circuit board; 10-power supply box; 11-fan; 100-power supply circuit; 200-MCU control circuit; 300-atomization driving circuit; 400-atomization control circuit; 201-water level detection circuit; 202-fan control circuit.

The realization of the purpose, functional features and advantages of the disclosure will be further described in combination with the embodiments and with reference to the attached drawings.

### DETAILED DESCRIPTION OF EMBODIMENTS

Next, the technical solutions in the embodiments of the disclosure will be described clearly and completely in combination with the attached drawings in the embodiments of the disclosure. Obviously, the described embodiments are only part of the embodiments of the disclosure, not all of the embodiments. Based on the embodiments of the disclosure, all other embodiments obtained by those skilled in the art without creative work belong to the protection scope of the disclosure.

It should be noted that in the embodiments of the disclosure, all directional indications (such as up, down, left, right, front, rear . . . ) are only used to explain the relative position relationship and movement between components under a specific attitude (as shown in the attached drawings). If the specific attitude changes, the directional indication will change accordingly.

In the disclosure, unless otherwise specified and limited, the terms “connected”, “fixed” and so on should be understood in a broad sense. For example, “fixed” can be fixed connection, removable connection or integrated; or can be mechanical connection or electrical connection; or can be directly connected or indirectly connected through an intermediate medium; or can be the connection within two elements or the interaction relationship between two elements, unless otherwise clearly defined. For those skilled in the art, the specific meaning of the above terms in the disclosure can be understood according to the specific situation.

In addition, in the disclosure, the description of “first”, “second” and the like is only for the purpose of description, and cannot be understood as indicating or implying its relative importance or implicitly indicating the number of indicated technical features. Thus, the features defining “first” and “second” may include at least one of the features explicitly or implicitly. In addition, the meaning of “and/or” in the full papers includes three parallel schemes. Taking “A and/or B” as an example, it includes scheme A, or scheme B, or schemes A and B. In addition, the technical solutions between various embodiments can be combined with each other, but it must be based on the realization of those skilled in the art. When the combination of technical solutions is contradictory or impossible, it should be considered that the combination of technical solutions does not exist and is not within the protection scope required by the disclosure.

The disclosure provides an essential oil diffusion using device.

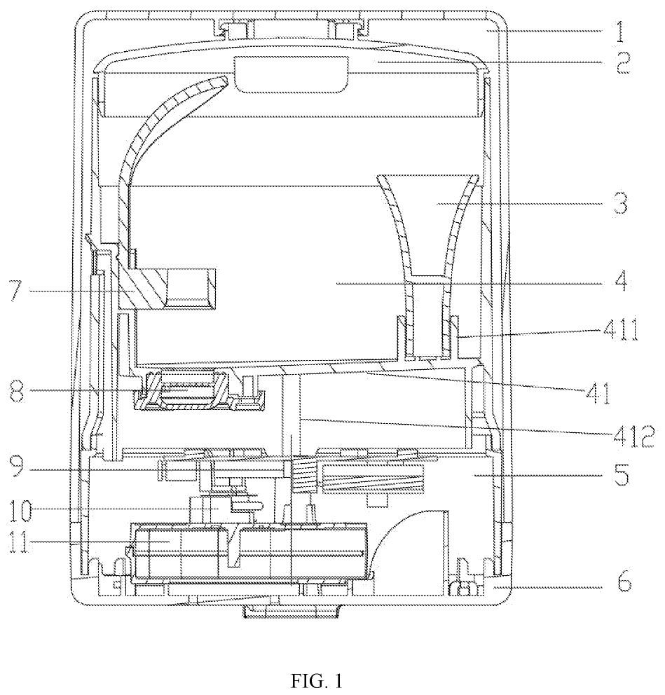

Referring to FIG. 1, in an embodiment, an essential oil diffusion using device includes a housing 1. A water tank 4 is arranged in the housing 1. A support frame 41 is arranged in the water tank 4. A side of the support frame 41 is provided with an essential oil pipe 3, another side of the support frame 41 is provided with a water stopping sheet 7, an atomizing sheet 8 is arranged at a bottom of the another side of the support frame 41. In an embodiment, the atomizing sheet 8 and the water retaining sheet 7 are arranged on a same side of the support frame 41.

A middle holder 5 is arranged below the support frame 41, and the middle holder 5 is a cavity. A fan 11 is arranged at a bottom of the middle holder 5, and a power supply box 10 is arranged at a top of the fan 11. In an embodiment, a power supply is arranged in the power supply box 10. An atomization control circuit board 9 is arranged between the power supply box 10 and a top of the middle holder 5. The atomization control circuit board 9 is provided with an atomization circuit.

In an embodiment, the atomization control circuit board 9 can be arranged at the top of the middle holder 5 and electrically connected to the power supply in the power supply box 10.

In an embodiment, the essential oil pipe 3 can be assembled on the water tank 4 in an aromatherapy machine. The water mist hit by the atomizing sheet 8 splashes into the essential oil pipe 3 through the water stopping sheet 7, and the mist in the water tank 4 is collected in the essential oil pipe 3, by collecting the water mist, the water level in the essential oil pipe 3 rises to make the essential oil overflow slowly, so as to delay the diffusion of essential oil and make the smell diffusion of the aromatherapy machine more lasting.

In an embodiment, a water stopping cover 2 is arranged at the top of the water stopping sheet 7.

In this embodiment, the water stopping cover 2 can better prevent the water mist from spreading out of the water tank 4, mix the water mist with the essential oil in the essential oil pipe 3, and make the diffusion of the essential oil smell more lasting.

In an embodiment, a side of the support frame 41 is further provided with an essential oil slot 411, and an end of the essential oil pipe 3 is arranged in the essential oil slot 411.

In an embodiment, the end of the essential oil pipe 3 is arranged in the essential oil slot 411, and the use of the essential oil pipe 3 is more safe, stable and reliable.

In an embodiment, the bottom of the support frame 41 is provided with a support rod 412, and the support frame 41 is connected to the middle holder 5 through the support rod 412.

In this embodiment, the support frame 41 is connected to the middle holder 5 through the support rod 412, thereby the support frame 41 is more stable, safer and reliable; and the essential oil pipe 3 of the support frame 41 is also more stable and emits the smell of essential oil more effectively.

In an embodiment, the bottom of the middle holder 5 is provided with a base 6, which is detachably connected to the housing 1.

In this embodiment, the base 6 is detachably connected to the housing 1, which makes the maintenance and use of the essential oil diffusion using device more convenient.

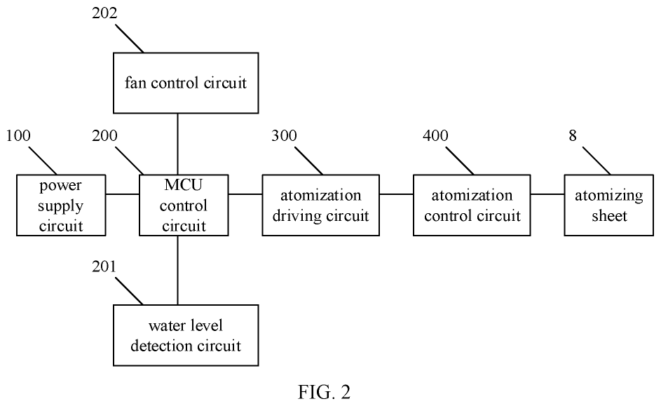

Referring to FIG. 2, in an embodiment, the atomization circuit includes a power supply circuit 100, a MCU control circuit 200, an atomization driving circuit 300, an atomization control circuit 400 and a fan control circuit 202. The power supply circuit 100 is configured to provide a power to the MCU control circuit 200. The MCU control circuit 200 is configured to control the atomization control circuit 400 through the atomization driving circuit 300. The atomization control circuit 400 is configured to control the atomization sheet 8. The MCU control circuit 200 is configured to control the fan control circuit 202. The fan control circuit 202 is configured to control the operation of the fan.

In an embodiment, The MCU control circuit 200 includes a water level detection circuit 201, which can detect the water level in the essential oil pipe 3 to ensure the more effective and lasting emission of the essential oil.

The disclosure can be used not only in the essential oil diffusion device, but also in ultrasonic atomization products such as a humidifier, and has a wide range of applications. The disclosure has a simple structure and is a technical upgrade in the field of essential oil diffusion.

The above description is only optional embodiments of the disclosure, which does not limit the scope of the disclosure. Under the inventive concept of the disclosure, the equivalent structural replacements made by using the contents of the specification and drawings of the disclosure, or directly/indirectly applied in other related technical fields, are included in the protection scope of the

What is claimed is:

1. An essential oil diffusion using device, comprising:
a housing,
a water tank, arranged in the housing;
a support frame, arranged in the water tank;
an essential oil pipe, arranged at a side of the support frame;
a water stopping sheet, arranged at another side of the support frame;
an atomizing sheet, arranged at a bottom of the another side of the support frame;
a middle holder, arranged below the support frame;
a fan, arranged at a bottom of the middle holder;
a power supply box, arranged at a top of the fan;
an atomization control circuit board, arranged between the power supply box and a top of the middle holder; and
an atomization circuit, arranged on the atomization control circuit board.

2. The essential oil diffusion using device according to claim 1, further comprising:
a water stopping cover, arranged at a top of the water stopping sheet.

3. The essential oil diffusion using device according to claim 2, further comprising:
an essential oil slot, arranged at the side of the support frame, and an end of the essential oil pipe being arranged in the essential oil slot.

4. The essential oil diffusion using device according to claim 3, further comprising:
a support rod, arranged at a bottom of the support frame, and the support frame being connected to the middle holder through the support rod.

5. The essential oil diffusion using device according to claim 4, further comprising:
a base, arranged at the bottom of the middle holder and detachably connected to the housing.

6. The essential oil diffusion using device according to claim 1, wherein the atomization circuit comprises a power supply circuit, a microcontroller unit (MCU) control circuit, an atomization driving circuit, an atomization control circuit and a fan control circuit;
wherein the power supply circuit is configured to provide a power to the MCU control circuit, the MCU control circuit is configured to control the atomization control circuit through the atomization driving circuit, the atomization control circuit is configured to control the atomization sheet, and the MCU control circuit is further configured to control the fan control circuit.

7. The essential oil diffusion using device according to claim 6, wherein the MCU control circuit comprises a water level detection circuit.
